(19) United States
(12) Patent Application Publication
(10) Pub. No.: US 2005/0196374 A1
(43) Pub. Date: Sep. 8, 2005

(54) Defecation deodorant

(76) Inventor: Shoji Ueda, Sakai-shi (JP)

Correspondence Address: JORDAN AND HAMBURG LLP, 122 EAST 42ND STREET, SUITE 4000, NEW YORK, NY, 10168, US

(21) Appl. No.: 10/955,341

(22) Filed: Sep. 29, 2004

(30) Foreign Application Priority Data
Mar. 5, 2004 (JP) 2004-62694

(51) Int. Cl. A61L 9/01; A61K 9/26

(52) U.S. Cl. 424/76.1

(57) ABSTRACT

An orally ingestible defecation deodarant reduces the production of intestinal gas and the production of malodors accompanying defecation or the emanation of intestinal gas, by absorbing malodor causing substances from the gastrointestinal tract; prevents the occurence of constipation; and adsorbs substances from the gastrointestinal tract that interfere with the absorption of orally administered drugs, pharmaceuticals, and other active ingredients from the gastrointestinal tract into the bloodstream and/or body. The defecation deodorant contains at least one adsorbent selected from a chelate compound, coconut vinegar, food fiber, gelatin, polyphenol, and a plant extract, on the surface of activated carbon.

### BACKGROUND OF THE INVENTION

1. Field of the Invention

The present invention relates to a defecation deodorant which upon oral ingestion into the body, particularly adsorbs and excretes bad smelling gas occurring and retaining in the intestines and bad smelling substance in feces, to adsorb and remove bad smells upon evacuation and farting.

2. Description of the Related Art

Due to economical advance after the Second World War, the eating habits of the Japanese are now very rich, and are so diversified as to be ridiculed as “satiation Japan” by many foreigners.

However, food circumstances supporting such satiation culture are transferred from grain/vegetable-directed eating habits (vegetarianism) to meat-directed eating habits (carnivorousness). It is said that meat contains nitrogen components and sulfur components in higher amounts than in grain and vegetables, and generates bad smelling gases and bad smelling substances such as ammonia, hydrogen sulfide, indole and skatole in the biodegradation process, and peoples hesitate to evacuate the vowels or to fart outside home, resulting in occurrence of health difficulties and in hesitation to go out. In addition, these bad smelling gases are retained in the intestines to cause growling in the stomach and expansion of the stomach to generate an unpleasant feel. The bad smelling substances are absorbed via digestive organs, particularly via the small and large intestines, into the body and accumulated in the body without excretion, which may result in health deterioration of which persons are unaware.

Particularly, the average longevity of the Japanese is drastically prolonged after the Second World War, and pharmaceutical preparations advance significantly together with the prolongation of longevity and fulfillment of medical services, and the amount of pharmaceutical preparations produced is increasing. Due to the unbalance between recent declining birthrate and aging, the necessity for nursing care for the elderly is increasing, and thus the education and securing of care workers are highly demanded in this society.

However, the elderly have a certain pharmaceutical preparation in maintaining their health and in treating diseases, but administration of the pharmaceutical preparation is accompanied generally by a very bad smell in evacuation or farting, resulting in hesitation of care workers to nurse those (e.g. the elderly) to be cared and nursed, which is a common worry among the care workers and those to be nursed.

Peoples loving animals or relieving loneliness attributable to a decreasing number of children keep pets such as dogs and cats often in the inside and the outside of a room, but there are not a few pet keepers who worry about a bad smell accompanying defecation of pets.

However, these worries or problems are not directly dealt with probably because these are hesitated to be directly addressed.

On the other hand, the influence of harmful substances contained in foods, such as heavy metals, organochlorine compounds, organotin compounds, mercury, PCB, TBT and dioxin on health cannot be negligible, and thus an adsorptive scavenger for harmful substances contained in foods etc. have been recently developed (for example, JP-A 09-075723 and JP-A 10-506775).

That is, an adsorptive scavenger for harmful substances disclosed in JP-A 09-075723 adsorbs and removes harmful substances adhering to, or contained in, foods etc. or occurring in the digestive system by directly bringing an adsorptive scavenger for harmful substances, comprising activated carbon having a particle diameter of 5 to 10 μm dispersed in an amount of 0.02 to 90 wt % in a physiologically acceptable gel-like dispersant, into contact with foods etc. or by administering the scavenger directly into the digestive system.

An adsorbent disclosed in JP-A 10-506775 is produced by applying a gel-like substance onto an adsorbent such as activated carbon and then subjecting it to freezing treatment.

The activated carbon is an adsorbent which is made of wood, sawdust, dry-distillated wood, charcoal, coconut shell or lignin as a starting material (activated carbon material) which has been subjected to special treatment (activating treatment) to improve its ability to adsorb gas, coloring matter etc. The activated carbon has a relatively high ability to adsorb fine-particle components and is inexpensive, and is thus the most frequently used adsorbent at present in the field of deodorants in refrigerators and shoe cupboards, filters in air cleaners, and other deodorant and adsorptive products.

However, these adsorptive scavengers for harmful substances cannot adsorb a wide variety of harmful substances so that without being adsorbed, some heavy metals and harmful substances are accumulated in the body, and such adsorptive scavengers are extremely inferior in the ability to absorb bad smelling gases and bad smelling substances generated by biodegradation, such as ammonia, hydrogen sulfide, indole and skatole.

The present inventors made extensive study for solving the problem described above, and as a result they arrived at development of a defecation deodorant characterized in that a defecation deodorant having at least one member selected from plant extracts such as a chelate compound, coconut vinegar, food fiber, gelatin, polyphenol and an aloe extract carried on the surface of activated carbon is contained as an active ingredient.

That is, the present inventors found that various bad smelling gases and bad smelling substances, aside from various harmful substances, are hardly dealt with by mere ingestion of the conventional absorptive scavengers, but a defecation deodorant comprising a specific substance such as a chelate compound or coconut vinegar carried on the surface of activated carbon can improve its ability to adsorb the bad smelling gases and bad smelling substances rather than various harmful substances, and this defecation deodorant can be a deodorant capable of adsorbing the bad smelling gases and bad smelling substances.

The present inventors found that the defecation deodorant comprising such adsorbent as the major active ingredient can be orally ingested to adsorb the bad smelling gases thereby preventing the gases from being retained in the intestines, resulting in significant reduction in farting and leading to pleasant daily life, and also that the bad smelling substances are adsorbed into the defecation deodorant in the digestive system, particularly in the small and large intestines, resulting in generating no or less bad smell upon defecating, thus letting peoples easily go out and fulfilling pleasant nursing services for the elderly by care workers.

A conventional adsorbent comprising activated carbon has strong adsorptivity, and thus adsorbs water or adsorbs a bifidobacterium in the intestine thus inhibiting the intestinal regulating action, to cause constipation, and upon administration together with a pharmaceutical preparation, adsorbs an active ingredient in the pharmaceutical preparation, to eliminate the efficacy, thus making it impossible to expect a desired therapeutic effect. In this administration, therefore, considerable attention should be paid.

The present inventors found that when activated carbon is to be produced, the size of superfine pores in activated carbon is changed by a 2-stage water steam activation treatment, whereby the activated carbon can change the ability to adsorb bad smelling substances and/or bad smelling gases, or can regulate the ability to adsorb a bifidobacterium, to eliminate occurrence of constipation, and upon administration together with a pharmaceutical preparation, can prevent an active ingredient in the pharmaceutical preparation from being adsorbed into the activated carbon, to achieve a desired therapeutic effect, and can thus be administered safely.

### SUMMARY OF THE INVENTION

The present invention was made on the basis of the foregoing findings, and the object of the present invention is to provide a defecation deodorant which upon oral digestion, adsorbs bad smelling gases to prevent the gases from being retained in the intestines, resulting in significant reduction in farting and leading to pleasant daily life, and the bad smelling substances are adsorbed into the defecation deodorant in the digestive system, particularly in the small and large intestines, resulting in generating no or less bad smells upon defecating, thus letting peoples easily go out and fulfilling pleasant nursing services for the elderly by care workers, and can further control the adsorptivity to prevent constipation and prevent the adsorption of an active ingredient in a pharmaceutical preparation.

To solve this problem, the defecation deodorant of the present invention is characterized in that an adsorbent having at least one member selected from a chelate compound, coconut vinegar, food fiber, gelatin, polyphenol and a plant extract carried on the surface of activated carbon is contained as a major active ingredient.

That is, at least one member selected from a chelate compound, coconut vinegar, food fiber, gelatin, polyphenol and a plant extract (hereinafter referred to as the specific substance) is carried on the surface of activated carbon thereby forming an adsorbent capable of controlling the adsorptivity of the activated carbon and adsorbing various bad smelling gases and bad smelling substance generated in the biodegradation process.

Upon oral administration, the defecation deodorant comprising the adsorbent as the major active ingredient adsorbs bad smelling gases to reduce farting significantly, or adsorbs bad smelling substances so that no or less bad smell is generated in defecation.

In the present invention, the terms “major active ingredient” is intended to mean that the active ingredient can be used alone as the defecation deodorant or may further carry nutrients, vitamins etc.

Hereinafter, the defection deodorant of the present invention is described in more detail.

The activated carbon used in the defecation deodorant of the present invention is not particularly limited, and usual activated carbon used as an industrial catalyst carrier or a deodorant, in purification of an objective substance, in recovery of an organic solvent, etc.

The starting material of the activated carbon is not particularly limited, and known activated carbon materials such as wood, sawdust, dry-distillated wood, charcoal, coconut shell and lignin can be preferably used, among which coconut shell which is easily available, inexpensive and stable in adsorption performance is particularly preferably used.

In the present invention, the activated carbon is preferably the one having superfine pores whose size was changed by subjecting the activated carbon to a 2-stage water steam activation treatment, and such activated carbon can change the ability to adsorb bad smelling substances and/or bad smelling gases or regulate the ability to adsorb a bifidobacterium, to eliminate occurrence of constipation, and upon administration together with a pharmaceutical preparation, can prevent an active ingredient in the pharmaceutical preparation from being adsorbed into the activated carbon, to achieve a desired therapeutic effect, and can thus be administered safely.

Generally, activated carbon is produced by milling charcoal or the like, then sifting it, activation by baking with high-temperature water steam, washing it with an acid such as hydrochloric acid or nitric acid to remove ashes, washing with water, treatment with sodium hydroxide, washing with water, drying, milling and then sifting. When the activated carbon is produced in this manner, relatively uniform superfine pores are formed to express adsorptivity higher than necessary, resulting in generating the above-described disadvantage

Preferably, to solve this problem, the activated carbon is activated by baking with high-temperature water steam in the process described above, and then cooled to room temperature, aged for several days to stabilize superfine pores, and then baked again with high-temperature water steam to change (i.e. increase) the size of some superfine pores, thus changing the ability to adsorb bad smelling substances and/or bad smelling gases, controlling the ability to adsorb a bifidobacterium, and controlling the ability to adsorb an active ingredient in a pharmaceutical preparation.

In the defecation deodorant of the present invention, at least one member selected from a chelate compound, coconut vinegar, food fiber, gelatin, polyphenol and a plant extract is carried on the surface of such activated carbon, whereby the adsorptivity of the activated carbon can be controlled, and various bad smelling gases and bad smelling substances generated in the biodegradation process can be efficiently adsorbed.

In the present invention, the specific substance carried on the surface of the activated carbon has a special skeleton or functional groups, and can, efficiently and synergistically with the activated carbon, adsorb various bad smelling gases and bad smelling substances generated in the biodegradation process.

The chelate compound used in the defecation deodorant of the present invention is not particularly limited insofar as the bad smelling gases and bad smelling substances can be efficiently adsorbed, and because the type of bad smelling gas and bad smelling substance to be captured is varied depending on the type of chelate forming group, the chelate compound is suitably selected depending on the intended use.

Specific examples of such chelating groups include —CSNH₂, —CSNHNH₂, —NHCSNHNH₂, —NHCSNH₂, iminodiacetic group, —(NHCH₂CH₂)—NH₂, >NCHRCOOH, >C═NOH, —C(NH₂)=NOH, >NCH₂—(C(OH)n—H, >NCH₂PO₃H₂, —C(NHOH)═O, —COCH₂—CO—, ═COH—CO—and —(C(OH)H)n—CH₂OH etc. Particularly compounds having —CSNH₂, —CSNHNH₂, —NHCSNHNH₂ or —NHCSNH₂, can be mentioned, and specific chelate compounds include EDTA, thiosemicarbazide, thiocarbazide, thiourea etc.

In the present invention, any one of these chelate compounds can be used singly, but a plurality of chelate compounds are used particularly preferably because various kinds of bad smelling gases and bad smelling substances can be adsorbed.

In the defecation deodorant of the present invention, the plant extract refers to materials obtained by extracting plants with a solvent, or to materials obtained by subjecting plants to distillation such as vacuum distillation or water steam distillation or to materials obtained by squeezing plants, and specifically, mention is made of at least one member selected from essential oil, gum resin, gum arabic, licorice extract, pop extract, aloe extract, malt extract, tea or mate extract, glycyrrhizin, garlic extract, oleoresin extract, natural balsam, pectin, pectate, pectininate and Otane carrot extract, and two or more thereof are preferably carried in relation with other substances to be carried.

In the present invention, at least one member selected from food fiber collected from coconut water or food fiber collected from aloe, and polyphenol having a catechin skeleton, are particularly preferable because they have a special skeleton or functional groups and exhibit an extremely high ability to adsorb various bad smelling gases and bad smelling substances generated in the biodegradation process.

In the defecation deodorant of the present invention, an adsorbent having the specific substance carried on the surface of the activated carbon is contained as the major active ingredient, and the method of carrying the specific substance is not particularly limited; for example, the activated carbon may be immersed in a dispersion or solution of the specific substance in a solvent, or the solution or dispersion may be sprayed on, or adsorbed into, the activated carbon and then dried by vacuum drying under conditions where the specific substance is not denatured or decomposed.

In the present invention, the amount of the specific substance to be carried on the activated carbon is not particularly limited and is determined suitably depending on the type of the specific substance or the type of activated carbon or properties thereof, but generally the amount of the specific substance to be carried on 100 parts by weight of activated carbon is preferably 0.1 to 10 parts by weight, more preferably 0.5 to 7.5 parts by weight, still more preferably 1.0 to 5.0 parts by weight.

When the amount of the specific substance to be carried on 100 parts by weight of activated carbon is less than 0.1 part by weight, the amount is too low to achieve desired absorptivity, while when the amount of the specific substance to be carried on 100 parts by weight of activated carbon is higher than 10 parts by weight, the amount is so high that the specific substance is wasteful and the absorptivity of the activated carbon may be hindered, being not preferable.

Because various kinds of bad smelling gases and bad smelling substances generated in the biodegradation process are generated in the digestive system, particularly in the small and large intestines, it is preferable that the defecation deodorant of the present invention is rendered most active in the intestines.

In the defecation deodorant of the present invention, therefore, the defecation deodorant is preferably encapsulated in an enteric capsule, and by this constitution, the orally ingested defecation deodorant of the present invention is protected with the enteric capsule until it reaches the intestine, and thus the activity is not reduced or lost, and upon reaching the intestine, the enteric capsule is dissolved to exhibit an active adsorptive action on bad smelling gases and bad smelling substances.

In the present invention, the method of carrying the defecation deodorant encapsulated in an enteric capsule, on the surface of activated carbon, is not particularly limited, but generally the defecation deodorant, together with the specific substance, may be carried on the surface of activated carbon, or may be dispersed and compounded in a gel-like material described later.

Preferably, the defecation deodorant of the present invention is further compounded with an antiflatulent agent, and by this constitution, the living body can be maintained in a normal state, and can prevent occurrence of constipation.

Such antiflatulent agent is not particularly limited, but generally food fiber or the like can be mentioned, and particularly bifidobacteria encapsulated in an enteric capsule are desirable because they regulate the living body in a more normal state and prevent occurrence of constipation.

In the present invention, the method of carrying the antiflatulent agent on the surface of activated carbon is not particularly limited, but generally the antiflatulent agent may be carried together with the specific substance on the surface of activated carbon or by dispersing and incorporating it into a gel-like material described later or by encapsulating it in an enteric capsule.

Because the defecation deodorant of the present invention wherein an adsorbent having the specific substance carried on the surface of activated carbon is contained as the major active ingredient is orally ingested, the outward appearance and a feel in eating and in swallowing should be improved.

In the defecation deodorant of the present invention, therefore, the activated carbon is preferably colored in gray or white by coating with a calcium salt of an organic acid, and by this constitution, the activated carbon can be colored in an arbitrary color such as pink, yellow etc. by an edible coloring matter.

The defecation deodorant of the present invention is ingested orally, and from the viewpoint of safety etc., the calcium salt of an organic acid in the defecation deodorant is preferably at least one member selected from calcium lactate and calcium alginate.

For improving a feel in eating and swallowing, the defecation deodorant of the present invention is coated preferably with a gel-like substance.

This gel-like substance is not particularly limited insofar as it is not toxic after administration into the digestive system and can wrap the adsorbent therein, and specific examples include alginic acid, sodium alginate, pectic acid, chitosan oxalate gel, starch such as corn starch etc., and carboxymethyl cellulose, calcium salts thereof, magnesium salts thereof and iron salts thereof, as well as gel-like foods such as jelly, konnyaku (gelatinous food made from devil's-tongue starch), agar and gelidium jelly.

When the surface of the activated carbon in the defecation deodorant of the present invention is coated with the gel-like substance, the activity of the activated carbon is lost, but the gel-like substance is eluted in the digestive system particularly during transfer to the small and large intestines, thus exposing the active defecation deodorant and permitting it to adsorb bad smelling gases and bad smelling substances efficiently.

The defecation deodorant of the present invention has the constitution described above, and upon oral ingestion, adsorbs bad smelling gases to prevent the gases from being retained in the intestines, thus reducing farting significantly and leading to pleasant daily life, and also the bad smelling substances are adsorbed into the defecation deodorant in digestive system, particularly in the small and large intestines, resulting in generating no or less bad smell upon defection, thus letting peoples easily go out and fulfilling pleasant nursing services for the elderly by care workers.

In the defecation deodorant of the present invention, the specific substance is carried on the surface of the activated carbon to control the adsorptivity of the activated carbon thereby preventing the occurrence of constipation and preventing an active ingredient in a pharmaceutical preparation from being adsorbed into the activated carbon, thus causing no problems in maintaining health and treating diseases.

The activated carbon used in the defecation deodorant of the present invention is the one having superfine pores whose size was changed by subjecting the activated carbon to a 2-stage water steam activation treatment, and such activated carbon can change the ability to adsorb bad smelling substances and/or bad smelling gases or regulate the ability to adsorb a bifidobacterium, to eliminate occurrence of constipation, and upon administration together with a pharmaceutical preparation, can prevent an active ingredient in the pharmaceutical preparation from being adsorbed into the activated carbon, to achieve a desired therapeutic effect, thus making the administration thereof safe.

### EXAMPLES

Hereinafter, the defecation deodorant of the present invention is described in more detail by reference to the Examples, but the present invention is not limited to the Examples.

### Example 1

(1) Preparation of Activated Carbon

Coconut shell was dried and baked to give an activated carbon material, and this material was then milled, sifted and subjected in a red-hot state to activation treatment with water steam at 1000° C. for 3 hours in a rotary kiln, then aged at room temperature for 2 days to stabilize superfine pores, and baked again with water steam at a high temperature of 1000° C. for 2 hours to endow the superfine pores with a varying size.

Then, ashes were removed by washing with an acid such as hydrochloric acid or nitric acid by a known method, then washed with water, treated with sodium hydroxide, washed with water, dried, milled and sifted to give powdery activated carbon (15- to 50-mesh size).

(2) Preparation of an Adsorbent (with a Chelate Compound Carried)

The activated carbon (100 g) obtained in (1) above was immersed in 10 wt % mixed solution of EDTA and thiocarbazide (1:1) for 6 hours, and the activated carbon was separated from the solution and dried, whereby an adsorbent having 1.75 g chelate compound carried on the surface of the activated carbon was obtained.

(3) Preparation of a Defecation Deodorant

The activated carbon obtained in (2) above was encapsulated in an enteric capsule, whereby the defecation deodorant of the present invention was obtained.

Then, the defecation deodorant obtained in (3) above and the activated carbon obtained in (1) above were used in a comparative test.

<Comparative Test 1>

60 g of cucumber slice (containing chlorothalonil (TPN) as an organochlorine agrochemical) was added to 300 ml acetone and homogenated for 5 minutes in a high-speed homogenizer.

The resulting homogenate was filtered, and the filtrate was divided into 3 test solutions (about 100 ml/test solution), and 1 g of the defecation deodorant of the present invention obtained in (3) above was added to one test solution; 1 g of the activated carbon obtained in (1) above was added to one of the other test solutions; and nothing was added to the last test solution. Then, each sample was stirred for 10 minutes.

Thereafter, each test solution was treated by a method described on pages 96 to 99 in “Medemiru Shokuhin Eisei Kensaho” (Food Sanitary Examination Method with Eyes), edited by Misao Haruda and published by Chuo Hoki Shuppan, and the concentration of the organochlorine agrochemical [chlorothalonil (TPN)] was quantified by gas chromatography.

The result indicated that the amount of chlorothalonil detected in the test solution to which the defecation deodorant of the present invention had been added was lower than the quantification limit of 0.001 ppm, while chlorothalonil was detected at a level of 0.826 ppm in the test solution to which the activated carbon had been added, and in the detection solution to which nothing had been added, chlorothalonil was detected at a level of 1.887 ppm.

<Comparative Test 2>

Three kinds of dog food were prepared by blending 100 parts by weight of dog food with 5 parts by weight of the defecation deodorant of the present invention in (3) above, or with 5 parts by weight of the activated carbon in (1) above, or with nothing, and each dog food was given to 3 dogs (Shiba dogs of the same age and weight) for 3 days, and feces excreted after the 3 days were colleted, and an ammonia gas generated from the feces was measured by a gas detection tube.

The result indicated that no ammonia gas was detected in the feces from the dog given the food compounded with the defecation deodorant of the present invention, ammonia gas was detected at the level of 35 ppm in feces from the dog given the food compounded with the activated carbon, and methane gas was detected at 100 ppm or more in feces from the dog given the food blended with nothing.

No bad smell was felt in the feces from the dog given the food compounded with the defecation deodorant of the present invention, a slight bad smell was felt in the feces from the dog given the food compounded with the activated carbon, and a considerable bad smell was felt in the feces from the dog given the food blended with nothing.

1. An orally ingestible defecation deodorant, comprising:
an at least one adsorbent, selected from the group consisting of:
a chelate compound, coconut vinegar, food fiber, gelatin, polyphenol, and a plant extract,
on a surface of activated carbon.

2. The defecation deodorant according to claim 1, wherein said activated carbon is activated by a 2-stage water steam activation treatment; and has superfine pores capable of adsorbing at least one of malodorous solid, liquid, and gaseous substances, and bifido bacteria, from a gastro-intestinal tract.

3. The defecation deodorant according to claim 1 or 2, wherein said food fiber is at least one selected from the group consisting of coconut water food fiber, and aloe food fiber.

4. The defecation deodorant according to claim 1 or 2, wherein said polyphenol has a catechin skeleton.

5. The defecation deodorant according to claim 1 or 2, encapsulated in an enteric capsule.

6. The defecation deodorant according to claim 1 or 2, further comprising an antiflatulent agent.

7. The defecation deodorant according to claim 6, wherein said antiflatulent agent comprises bifido bacteria, encapsulated in an enteric capsule.

8. The defecation deodorant according to claim 1 or 2, wherein said activated carbon is coated with a calcium salt of an organic acid.

9. The defecation deodorant according to claim 8, wherein said calcium said of an organic acid is at least one of calcium lactate and calcium alginate.
